(19) United States
(12) Patent Application Publication
(10) Pub. No.: US 2011/0152997 A1
(43) Pub. Date: Jun. 23, 2011

(54) DELIVERY SYSTEM FOR MULTIPLE STENTS

(76) Inventors: Daniel John Kelly, County Wicklow (IE); Ian Owens Pericevic, Leon (ES); Ghahi Saeid Kasiri, Wakefield, KS (US)

(21) Appl. No.: 12/737,052

(22) PCT Filed: May 29, 2009
(86) PCT No.: PCT/IE2009/000031

§ 371 Date: Feb. 18, 2011

Related U.S. Application Data
(60) Provisional application No. 61/129,110, filed on Jun. 5, 2008.

(51) Int. Cl. A61F 2/84

(52) U.S. Cl. 623/1.12

(57) ABSTRACT

A stent delivery balloon (1) is used to deploy a plurality of mini-stents (5) into a bodily lumen at a controlled spacing. The balloon (1) has formations (3) for retaining the stents in a designed position. The formations may comprise ridges (3), or retractable bridging elements, on the surface of the balloon (1) to control both the expansion of the multiple stents (5) from a single balloon (1) and also the coverage of the vessel wall following stenting.

### INTRODUCTION

Stenting has been used as a means to combat coronary artery disease since the 1980s and has proved to be a highly successful and low-cost alternative to coronary bypass. Many stents developed for the peripheral vessels were based on platforms designed for coronary applications and were not designed with the unique operating environment of peripheral arteries in mind. To this day, the success rate of peripheral stenting remains far below that of coronary stenting, with higher incidences of restenosis following treatment (Shouse et al. Endovascular Today, 2005; 4, 60-66; Matsi et al. 1995, Clin Radiol. 50(4):237-44; Mukherjee et al. 2001. Cleve Clin J Med. 68(8):723-33; Schillinger et al. 2006. N Engl J Med. 354(18):1879-88).

Peripheral arteries are highly flexible vessels which undergo various bending, twisting and torsion modes in multiple planes. Simultaneously, the relatively large diameters of peripheral vessels requiring stenting will mean a thicker vessel wall causing increased radial compression. Therefore, peripheral stents should allow maximum flexibility, whilst providing good support of the vessel wall and resisting radial forces. In terms of contemporary stent designs, this has proven difficult to achieve, requiring a trade-off between stent flexibility and wall support.

Traditional balloon expandable stents are used with high success rates in the vast majority of coronary procedures. However, they are not used in peripheral arteries as many of these arteries are subjected to a more extreme mechanical environment (i.e vessel bending due to joint flexion and extension, muscular forces etc.) than coronary arteries. Highly rigid balloon expandable stents tend to cause significant injury when implanted into such arteries, and tend to become permanently deformed and/or subject to fatigue failure. For these reasons, self-expanding stents are more commonly implanted into such arteries in the belief that they can overcome many of the problems associated with balloon expandable stents. However self-expanding stents provide less support and reliability in situ than classical balloon inflatable stents, not to mention less controllability during implantation. Generally, self-expanding stents are a less popular tool amongst interventional cardiologists. The patency rates of peripheral interventions are in general lower than those of coronary interventions.

In our PCT/IE2008/000116 we have described the use of a plurality of ‘mini-stents’ to provide flexibility to a stented vessel while maintaining radial support and wall coverage.

Controlling the longitudinal movement of the stent segments during delivery is necessary to prevent uncontrolled displacement of the stents from the delivery balloon during expansion. Some methods have already been proposed to deliver stents to a desired location. In these methods the stent movement on the balloon is generally limited to using friction or barriers.

When delivering multiple stents to the site of implantation, the distance between the segments after expansion is important. Correctly controlling the gap between the segments after deployment can reduce the risk of the restenosis (R. Tominaga, Am Heart J, 1992, 123, 21-28). Uniform support of a diseased artery with multiple stents also depends on accurately controlling the relative positioning of the stents in situ.

Due to the expansion and unfolding of a stent delivery angioplasty balloon during inflation, the stents tend to rotate during the deployment due to friction between the balloon and stent. This rotation of stent segments is undesirable as it can change the position of the multiple stent segments with respect to each other.

### STATEMENTS OF INVENTION

According to the invention there is provided a stent delivery system comprising:—
a plurality of separate or separable balloon expandable stent segments;
an expansible balloon on which the stent segments are mounted;
the balloon having a number of formations to control movement of the stent segments during deployment.

The invention also provides an expansible stent delivery balloon for reception of a plurality of separate or separable balloon expansible stent segments, the balloon having a number of formations to control movement of the stent segments during deployment.

In one embodiment the formations control the position of the stent segments on the balloon in a delivery configuration.

In one case the formations engage with at least some of the stent segments during deployment.

In one embodiment the formations engage with at least some of the stent segments in the delivery configuration.

In one case the balloon is folded in a delivery configuration and the formations are arranged such that the formations move only in a radial direction on expansion of the balloon. In one embodiment the formation are arranged such that the formations move only in a radial direction on expansion of the balloon.

There may be a plurality of circumferentially spaced-apart formations.

In one case the balloon has a longitudinal axis and there are a plurality of longitudinally spaced-apart formations.

At least some of the formations are integral with the balloon. Alternatively or additionally at least some of the formations are mounted on or mountable to the balloon.

In one embodiment at least some of the formations comprise protrusions extending radially outwardly of the balloon. The protrusions may extend radially outwardly of the balloon for a distance which is approximately the same as the thickness of a stent element.

In one embodiment the stent segments comprise a proximal segment, a distal segment and at least one intermediate segment. The segments may be inter-engaged at least in the delivery configuration. Formations may be located on the balloon to control the movement of at least the distal stent segment. Formations may be located on the balloon to control the movement of at least the proximal stent segment. The formations may be located on the balloon to control the movement of at least one of the intermediate stent segment(s).

In one embodiment in the delivery configuration the formations are all on the outer surface of the balloon folds or surface.

In one embodiment the height of the formations does not change significantly during expansion.

In one embodiment the multiple stent segments are separate segments which are not interlinked.

In another embodiment the stent segments have coupling parts for coupling of the segments, the segments being movable between:—
a collapsed delivery configuration in which the coupling parts of the segments are interengaged; and
a deployed configuration in which the coupling parts are disengaged.

In one case the stent segments have means to delay the disengagement of the coupling parts until the stent segments are close to the deployed configuration.

In one embodiment the coupling parts comprise a male part and a female part, the male and female parts of adjacent stent segments being interengaged in the collapsed delivery configuration and the male and/or female part comprising the delay means to delay the disengagement of the coupling parts until the stent segments are close to the deployed configuration.

The female part may comprise an axially extending passageway having an entrance to receive a corresponding axially extending male part of an adjacent stent segment, the delay means comprising interengagable mating parts on the male and female parts, the mating parts being spaced axially inwardly of the entrance to the passageway.

In one embodiment there are first mating parts and second mating parts which are axially spaced-apart along the passageway.

The second mating parts may be located at an end of the passageway remote from the entrance.

In one case the second mating parts comprise a head part and a socket part for engagement with the head part. The socket part may comprise a neck which is of reduced dimensions with respect to the head part for retaining the head part in the socket part.

In one embodiment the head part comprises a ball.

The head part may comprise at least one radially extending projection. Preferably the head part comprises a pair of oppositely directed projections.

In one case the projecting portion is of generally rectilinear shape.

In another case the projecting portion is of generally wedge shape.

The projecting portion may be of generally curvilinear shape.

In one embodiment the stent segments are designed so that the male and female parts undergo differential deformation and/or displacement during expansion.

One of the female part or male part may undergo deformation and/or displacement during expansion and the other of the male part or female part does not undergo significant deformation or displacement.

In one case the female part undergoes deformation and/or displacement during expansion and the male part does not undergo significant displacement or deformation.

In another case the male part undergoes deformation and/or displacement during expansion and the female part does not undergo significant displacement or deformation.

In another case both the male and the female parts undergo deformation and/or displacement during expansion.

In one embodiment in the collapsed configuration, the male part extends substantially fully into the female part. In the collapsed configuration, the male part may be configured to substantially fill the female part.

In one case the stent segment comprises a first set of strut elements and a second set of strut elements. The stent segment may comprise a first set of one or more link elements to link at least some of the first set of strut elements to at least some of the second set of strut elements.

The invention also provides an endoprosthesis comprising a plurality of axially arranged radially expandable stent segments, the segments having coupling parts for coupling of the segments, the segments being movable between:—
a collapsed delivery configuration in which the coupling parts of the segments are interengaged; and
a deployed configuration in which the coupling parts are disengaged,
wherein the segment comprises a first set of strut elements, a second set of strut elements, and a first set of one or more link elements to link at least some of the first set of strut elements to at least some of the second set of strut elements and wherein a link element is more flexible than a strut element.

The invention also provides a prosthesis in which a link element extends rather than compresses by virtue of the stent end being engaged with an adjacent stent for a significant part of the expansion process or by virtue of a stent end being engaged with a formation on the balloon.

In a preferred embodiment the link element is more flexible than the strut element.

The link element may extend in a non-straight manner between the first set of strut elements and the second set of strut elements. The link element may open up or elongate during expansion of the stent.

The link element may extend in a substantially “s”-shape.

The link element may extend in a substantially “w”-shape.

The link element may extend in a substantially “m”-shape.

The link element may extend in a substantially “v”-shape.

In one case a closed cell is defined between the first set of strut elements, the second set of strut elements, and the link elements.

The closed cell may be defined between two strut elements of the first set of strut elements, two strut elements of the second set of strut elements, and two link elements.

The closed cell may be defined between four strut elements of the first set of strut elements, four strut elements of the second set of strut elements, and two link elements.

Preferably the first set of strut elements and the second set of strut elements are connected by at least one link element in the circumferential direction.

In one embodiment at least part of the segment comprises a biodegradable material.

In another embodiment at least part of the segment comprises a radiopaque material.

In a further embodiment there is a coating around at least part of the segment.

The coating may comprise a biologically active agent.

The prosthesis is particularly suitable for use in a peripheral artery.

In a further aspect the invention provides a method for delivering a luminal prosthesis to a treatment site comprising:—
providing a delivery catheter of the invention with a plurality of radially expandable stent segments arranged axially on the delivery catheter, the stent segments having coupling parts which are interengaged;
delivering the catheter to a treatment site;
radially expanding all of the stent segments at the treatment site to a partially expanded configuration in which the coupling parts of the segments remain interengaged; and
further radially expanding all of the stent segments to a deployed configuration in which all of the coupling parts of the stent segments are disengaged.

The invention provides a stent delivery device comprising a balloon having formations placed on the balloon surface for deploying multiple stents to the desired position in the site of implantation with controlled stent segment movement during the expansion and controlled spacing after deployment.

The stent comprises a plurality of separate or separable stent segments and the balloon has formations for retaining at least some of the stent segments in a desired position on the balloon and controlling relative rotation and/or the minimum and/or maximum distance between the segments following expansion. The balloon may have formations for retaining each of the stent segments in a desired position, or for controlling the maximum movement of a given stent segment.

In one case the formations are located at certain spots at the ends of the balloon. These spots, termed RE (Radial Expansion) spots, are defined as points that generally only move radially during balloon expansion. In a second configuration, the formations are spaced-apart so that each stent segment is situated between the formations. In some cases, the ridges are not placed on RE spots.

The formations are generally solid with a height of similar order as the stent strut thickness. The ridges may be higher than the strut thickness. The formations can be in different shapes such as rectangular, circular, and the like.

The formations may be placed between the struts/segment or on the proximal and/or distal ends of the balloon or both. The formation may or may not be in contact with the struts in the crimped configuration.

The formations are integral to the balloon and some or all of them may be mounted to the balloon.

The formation may be made before or after crimping the stents into the balloon.

The formation can be made using different methods including the use of adhesives and/or heat treatment. The formations may be designed into the mould used to fabricate the balloon, so that the formations are present on the balloon surface after blow moulding.

The formations may used in conjunction with multiple interlocking stents. The interlocking system may control an intermediate stent segment positioning and/or the formations may control the movement and positioning of the terminal stent segment(s).

The formation may be used with multiple segments comprising link elements. In one case, the formations constrain the proximal and distal stent segments (the terminal segments on the balloon) in order to ensure the link elements within each stent segment elongates during expansion. Alternatively, in cases where multiple stents do not interlock in the crimped configuration, the formations may constrain or limit the movement of all stents on the balloon, and ensure that link elements elongate during stent expansion. Limiting foreshortening of multiple stents is important in controlling the spacing between stent segments following expansion.

In one aspect the invention provides a stent delivery device comprising a balloon having formations for delivering and deploying multiple stent segments with controlled spacing between the segments following delivery of multiple interlocked stents. The stent comprises a plurality of separate or separable stent segments and the balloon has formations for controlling the relative positioning of the segments during the expansion. The balloon may have formations for delivering the segments into the desired position.

The formations on the balloon may prevent the undesirable axial movements of stent segments.

In one case of the interlinked or interlocked stent segments the formations prevent the undesirable displacement of the terminal segments, and by virtue of the fact that the multiple stents are interlocked until near the end of the expansion process, the formations in this case control the displacements of every stent segment on the balloon.

The formations may act as part of a stent-balloon mating system to interlock the segments to the balloon during delivery and expansion/deployment.

In one case the balloon has a plurality of formations which may or may not be in contact with stent struts at the beginning of the expansion process.

The formations may be located on the RE spots of the balloon.

In one case the formations are spaced-apart in between the segments along the balloon.

The formations thickness is typically of the same order as the thickness of the struts. The thickness of the formations on the balloon may/may not change during the delivery and expansion.

The formations/ridges on the balloon are used with crimped stents. The stents may be crimped onto the balloon after the formation is mounted on the balloon.

The formations may be mounted on the balloon after crimping the stents.

The formations may be used to control the rotation of the whole stent by controlling the rotation of the end stent segments.

In our PCT/IE2008/000116, the entire contents of which are incorporated herein by reference, we describe interlocking systems between multiple stent segments. The delivery system of the invention may be used with such stent segment assemblies.

This present invention uses solid ridges on certain points on the balloon surface to control the distance between the multiple stent segments in the expanded configuration and the rotation of the stents. These ridges also function to help prevent the uncontrolled longitudinal movement of the entire stent system during expansion.

This invention also describes the use of ridges on the balloon surface to control the movement of multiple stents, which in crimped configuration where interlocked, but by virtue of their design will unlock at some point during expansion process. At this point of unlocking, the ridges on the balloon control the relative positioning of the multiple stents as they move and foreshorten on the balloon.

The ridges may control the axial movement of the segments, especially the terminal segments in both directions. Controlling the movement of the terminal section prevents uncontrolled movement of the entire stent system along the balloon surface during delivery. The ridges can also be used to constrain the movement of the terminal stent segments toward the middle part of the balloon. This undesirable movement is due to opening of the proximal and distal sides of the balloon first which tends to push the stent segments towards the middle of the balloon. This can alter the spacing between the terminal segments on each side of the balloon.

A link element may be used in order to reduce the foreshortening of a given stent segment. The link element is straightened in each of the multiple stents during the deployment due to the interlocking system between adjacent stents, which effectively means the stent segments act as a single unit during expansion. In one aspect of the invention the displacement of the terminal stent is controlled in both directions.

### BRIEF DESCRIPTION OF THE DRAWINGS

The invention will be more clearly understood from the following description of an embodiment thereof, given by way of example only, with reference to the accompanying drawings, in which:—

FIG. 1 is a cross sectional view of wrapped balloon of a stent delivery system of the invention with regular folding, having ridges located on the outer surface;

FIG. 2 is a cross sectional view of a further balloon of the invention;

FIG. 3 is a side elevational view of the inflated balloon of FIG. 2;

FIG. 4 is an end view of the balloon of FIG. 3;

FIG. 5 is a cross sectional view of a balloon of the invention in a regular folded configuration with ridges on radial expansion (RE) spots to control stent movement;

FIG. 6 is a cross sectional view of a four folded symmetrically opening folded (SOF) balloon with ridges on the RE spots to control stent movement;

FIG. 7 is a cross sectional of a three folded SOF balloon of a stent delivery system with ridges on the RE spots;

FIG. 8 is a cross section of a three folded SOF balloon with ridges on two folds;

FIG. 9 is a cross section view of an SOF balloon with large formations on the RE spots;

FIG. 10 is a cross section view of a four folded balloon in the wrapped configuration with double folding of the balloon wings prior to wrapping in which ridges are located on the RE spots;

FIG. 11 is a side view of an expanded four fold balloon having formations;

FIG. 12 is an end view of the balloon of FIG. 11;

FIG. 13 is a side view of a three fold balloon having formations;

FIG. 14 is an end view of the balloon of FIG. 13;

FIG. 15 (a) to (f) are cross sectional views of various ridges for a balloon of the invention;

FIGS. 16(a) and 16(b) are cross sectional views of various couplings;

FIG. 17 is an isometric view of part of a mini-stent that may be used in the invention;

FIG. 18 is a side view of another balloon of the invention;

FIG. 19 is an elevational view of a balloon of the invention with multiple stents in place;

FIG. 20 is an elevational view of another balloon of the invention with multiple stents in place;

FIG. 21 is an elevational view of a further balloon of the invention with stents comprising link element in place;

FIG. 22 is an elevational view of another balloon of the invention with multiple stents having link elements in place;

FIG. 23 is an elevational view of a still further balloon of the invention;

FIG. 24 is a side elevational view of another balloon of the invention with multiple stents;

FIG. 25 is a side view of multiple interlocking stents crimped onto a single balloon constrained by ridges at the ends of the balloon to prevent axial movement of the stent along the balloon;

FIG. 26 is a side view of multiple interlocking stents crimped onto a single balloon with ridges near the balloon ends to prevent axial movement of the stent in both directions axially;

FIG. 27 is a side view and a cross section of an SOF balloon having ridges to control the movement of the terminal stent segments;

FIG. 28 is a side view of multiple interlocking stents with ridges controlling the axial movement of the middle of one terminal stem segment and the end of an opposite terminal stent segment;

FIG. 29 is a side view of multiple interlocking stents crimped onto a balloon with ridges acting to constrain the stent segment until such a point in the expansion process where the stent opens sufficiently to move past the ridge;

FIG. 30 illustrates an alternative interlocking stent system in which the stems will disengage earlier in the expansion process and where the ridges on the balloon control axial stent movement;

FIG. 31 illustrates an alternative balloon formation to control the axial movement of a stent segment in both directions;

FIG. 32 is a side view of multiple interlocking stents crimped onto a single balloon constrained by ridges at the ends of the balloon to prevent axial movement of the stem along the balloon, where the stem segments have link elements in their body that may straighten during expansion and hence limit foreshortening;

FIG. 33 is a side view of balloon having ridges at the end to prevent the movement of the terminal segments in both directions; and

FIG. 34 illustrates an alternative position for the ridges on a balloon in which the stent segments have link elements.

### DETAILED DESCRIPTION

The invention provides a stent delivery balloon 1 to deploy a plurality of separate ‘mini-stents’ 5, which may or may not be interlocked/interlinked in the crimped configuration, into a bodily lumen at a controlled spacing. The balloon has formations for retaining the stents in a designed position, or for controlling the movement of the stents along their delivery balloon. The formations may comprise ridges 3, or retractable bridging elements, on the surface of the balloon 1 to control both the expansion of the multiple stents 5 from a single balloon 1 and also the coverage of the vessel wall following stenting.

In one case the ridges 3 are integral parts of the delivery balloon surface. They are designed and positioned in such a way as to engage with the mini-stents 5 and restrict their translation and rotation with respect to the balloon surface until the balloon 1 has been fully inflated. When the mini-stents 5 have been delivered to the correct locations on the vessel wall, retraction of the balloon 1 is initiated, in a similar manner to a conventional delivery balloon/stent system. By this stage the mini-stents 5 will have undergone a significant amount of plastic deformation which will keep them firmly in contact with the vessel artery wall. When the balloon 1 is deflated, the ridges 3 are no longer engaged with the mini-stents 5, and the mini-stents can function as a single and highly flexible entity. In certain cases, the ridges 3 may be regarded as retractable bridge elements that ensure that the stent segments 5 act as a single system during expansion, and as individual stents after deflation.

In another case, the multiple stents are designed to interlock in the crimped configuration on the balloon. The stems are designed to separate late in the expansion process. By controlling when the stents separate, it is possible to control the final spacing of the stents. By positioning ridges at the proximal and distal ends of the balloon, it is possible to also control the longitudinal movement of the terminal segments that by virtue of their position are not engaged with other stent segments at both ends. The formations which are placed at the proximal and distal ends of the balloon to prevent the undesired movement of the terminal stent segments, may in turn control the displacement of middle segments of the stent that are directly or indirectly engaged with the terminal stent segments.

When the folded balloon is being inflated, due to the folding of the balloon there are many points on the balloon that undergo circumferential displacement as the balloon expands radially. Depending on how the balloon is folded, there may also be points that generally only move radially during expansion. For example, a four fold balloon can have four of these regions in each cross section (see FIG. 5-10). We call these regions “Radial Expansion” spots (RE). RE (Radial Expansion) spots are defined as points that generally only move radially during balloon expansion.

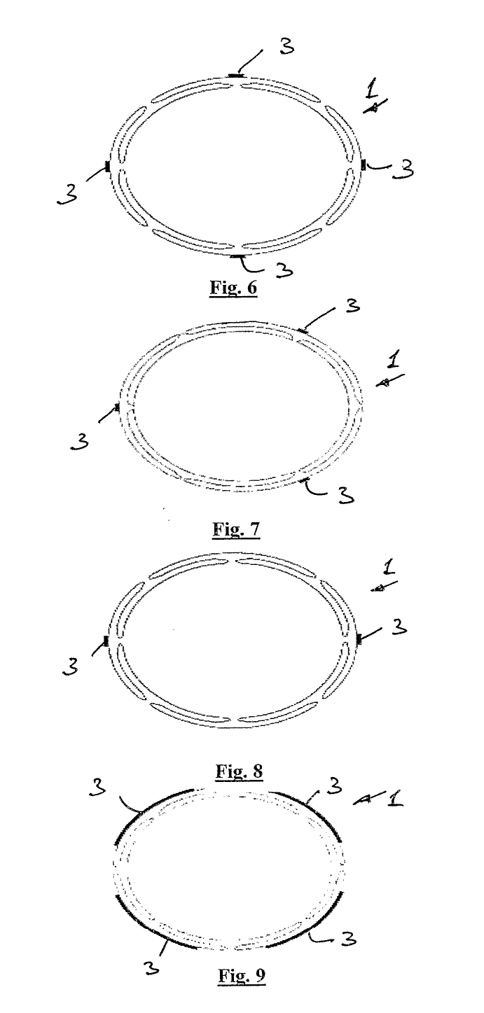

The RE spots depend on the folding method. For regular folding (FIG. 5) the RE spots are located on the inner layer of the balloon. In the Symmetric Opening Folded (SOF) balloon, the RE spots are located on the outer layer (FIG. 6-9). The region that undergoes only radial expansion on a SOF balloon is wider circumferentially compared with the regular folding. This is an advantage which provides a larger surface for making the formations. In the balloon with the larger ratio of inflated diameter to folded diameter, extra folding may be used to ensure the RE spot is accessible (see FIG. 10).

There are many different folding patterns which may be used with this invention. Referring to FIG. 1 in an embodiment of the invention, the ridges 3 may have a circumferential orientation. The open outer uncovered portions of the balloon wall are provided with ridges 3. In such a configuration the motion and positioning of the stents 5 is controlled by placing them within the areas on the balloon 1 bound by the circumferential ridges 3. During the inflation of the balloon 1 in such an embodiment the ridges 3 will follow the path of the unfolding balloon wall, which will not be directly radially outward but may also move circumferentially during expansion. In this case the balloon 1 has four folds 2 and four circumferential ridge locations 3 but may also move circumferentially during expansion.

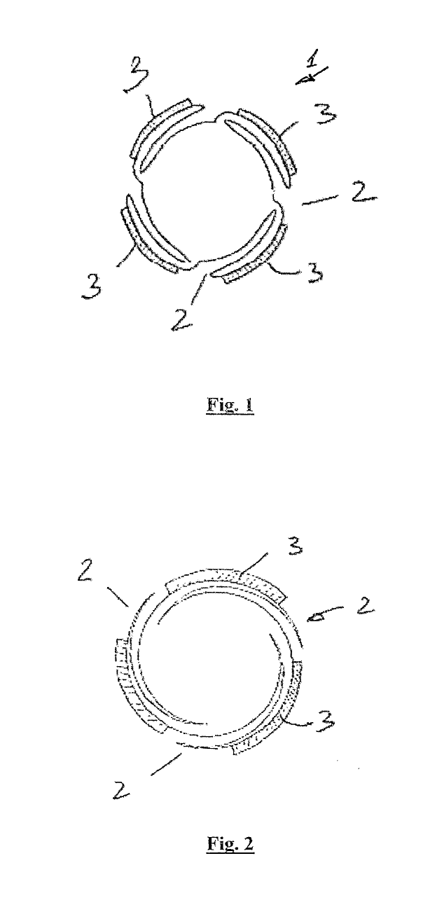

In FIG. 2 a ridged balloon is shown to represent that major overlapping of folds 2 occurs. It is also possible to apply ridges 3 with a circumferential orientation, as illustrated.

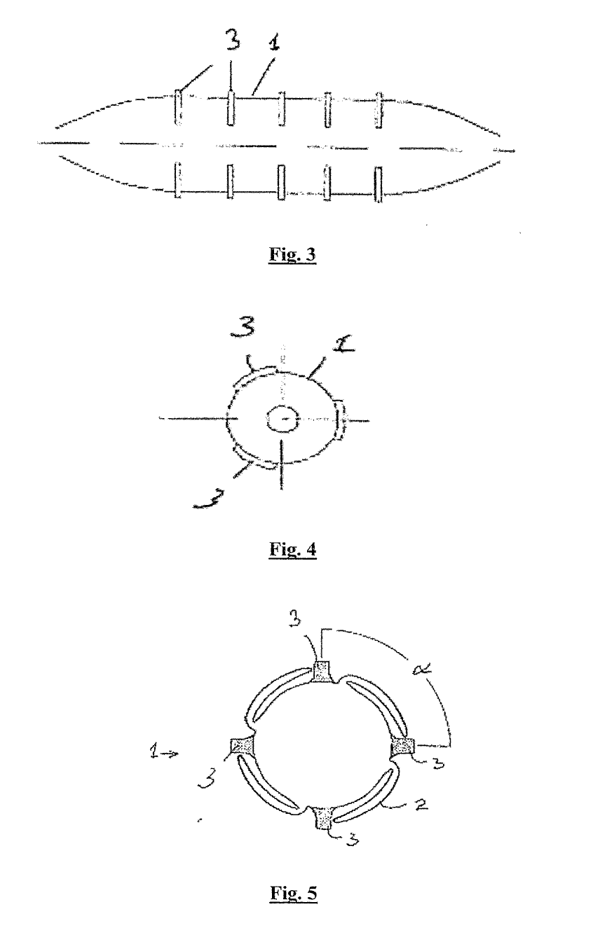

FIGS. 3 and 4 are views of a balloon similar to FIG. 2 but the balloon has a three folds 2 and three circumferential ridge locations of five ridges 3 each. The ridges 3, in this case, are spaced-apart equally around the circumference of the balloon 1 by an angle of 90° (alpha). The balloon 1 is folded in such a way that the outer surface of the ridges 3 is not covered by the balloon surface when the balloon is in a wrapped configuration. The wrapping method illustrated in FIG. 1 is just one of many possibilities to insure that undesired coverage of the ridge outer surface does not occur, whilst allowing sufficient balloon surface for the balloon 1 to expand to its nominal diameter during inflation. In this case, the ridge 3 is the part of the balloon which will interact with the mounted mini-stent 5 (or mini-stents) in such a way as to prevent axial motion of the mini-stent 5 when crimped and during inflation, whilst simultaneously regulating the spacing between mini-stents 5.

In FIG. 5 the cross-section of a balloon 1 in a folded configuration is illustrated. The balloon 1 has four folds 2 and four ridge locations 3 (hatched area) protruding from its surface. In addition finite element analysis shows that in such a configuration the ridges 3 will tend to predominantly move radially outward from the centreline (mandrel) of the balloon 1, and will allow the folds 2 of the balloon 1 to unfold without restriction to the balloon nominal diameter. Such a radial motion will ensure the minimum amount of undesirable stent motion and deformation during deployment due to interactions between the stents 5 and the ridges 3. In general, the number of ridge locations 3 a balloon 1 may have will be determined by the number of folds 2 (commonly two to eight), and hence the number of locations that are free to expand radially outward. The number of ridges 3 can vary, depending on requirements. The ridge 3 may be any kind of element protruding from the balloon wall surface. Alternatively there may be indentations on the balloon surface, which may be created during the manufacturing of the balloon (i.e. the moulding process). Alternatively elements 10 may be attached using adhesives or heat welding or otherwise as illustrated for example in FIG. 15.

FIG. 6 is another possible folding for the balloon. The RE spots on this SOF balloon are on the outer surface and more generally more accessible. Therefore a larger part of the balloon undergoes radial expansion compares to the folding in FIGS. 1,2. The balloon has four folds and the ridges are placed on all four folds.

FIG. 7 shows a SOF balloon with three folds with ridges fowled on each fold. The ridges can be located axially along the length of the balloon if necessary.

FIGS. 8 and 9 are four folded SOF balloons. In FIG. 8 the ridges are placed on two opposite folds. In FIG. 9 the ridges on the four folds of the balloon are longer in length.

FIG. 10 is another folding for the balloon. In this case an extra fold was used in each balloon wing in order to prevent the overlapping of balloon wings and keep the RE spots uncovered. This folding is especially useful when the final expanded diameter of the balloon is 3 or more times larger than its wrapped diameter. The balloon has four folds and the ridges have been placed on the RE spots.

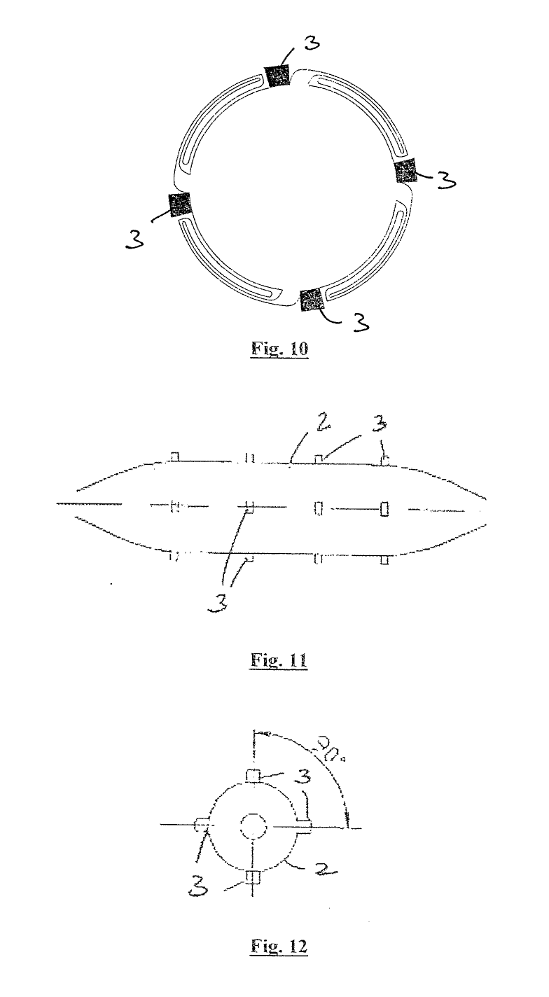

Referring in particular to FIGS. 11 and 12 there is illustrated one potential axial arrangement of the ridges 3 on the balloon 1. In this case, the balloon 1 has four ridge locations 3 spaced-apart equally around the balloon circumference at (90°) spacing. Each of the ridge locations spaced-apart has four rectangular ridges 3 that are equally spaced-apart. Many potential combinations of axial ridge location and size are possible, depending on the number and geometry of mini-stents 5 to be used.

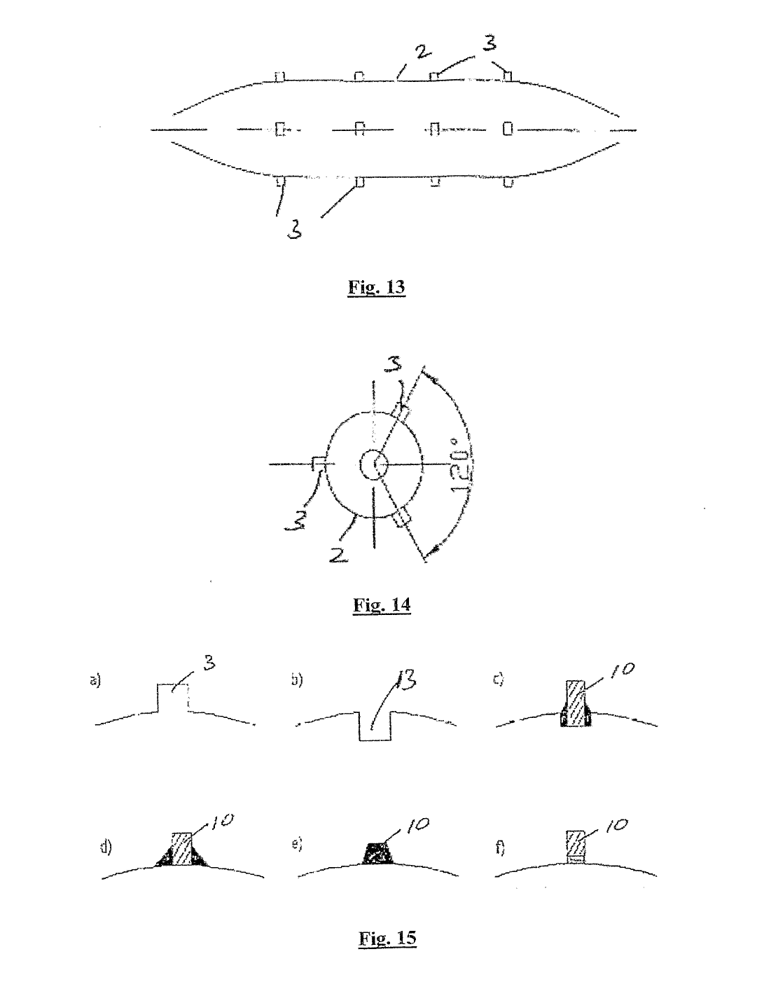

Referring to FIGS. 13 and 14 in an alternative configuration, there are three ridge locations (120° angle) around the circumference with four ridges 3 each. The axial alignment may remain the same.

Referring to FIG. 15 several alternatives to the balloon surface ridges and how those ridges are manufactured are illustrated. Those alternatives include, but are not limited to, the following: a) ridge 3 moulded with balloon during blow moulding or otherwise; h) channel 13 moulded with balloon; c) channel moulded with balloon, ridge made from a separate component 10 inserted into channel and connection formed with adhesive; d) ridge made from separate component 10 connected to balloon surface with adhesive; e) ridge 3 created solely by use of adhesive; and f) ridge made from separate component 10 heat welded onto balloon surface.

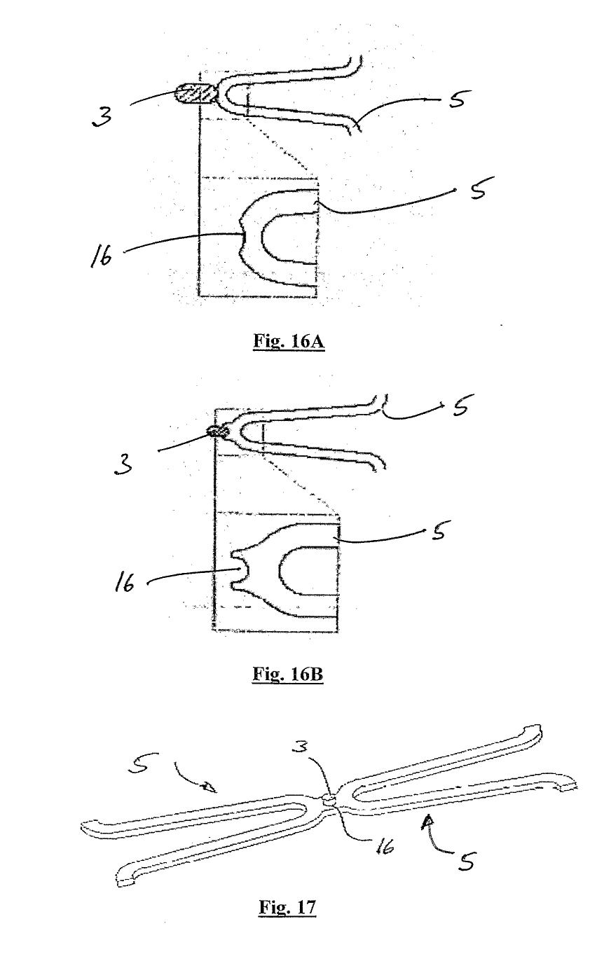

In FIG. 16 examples of coupling points designed on mini-stent segments to facilitate coupling are shown.

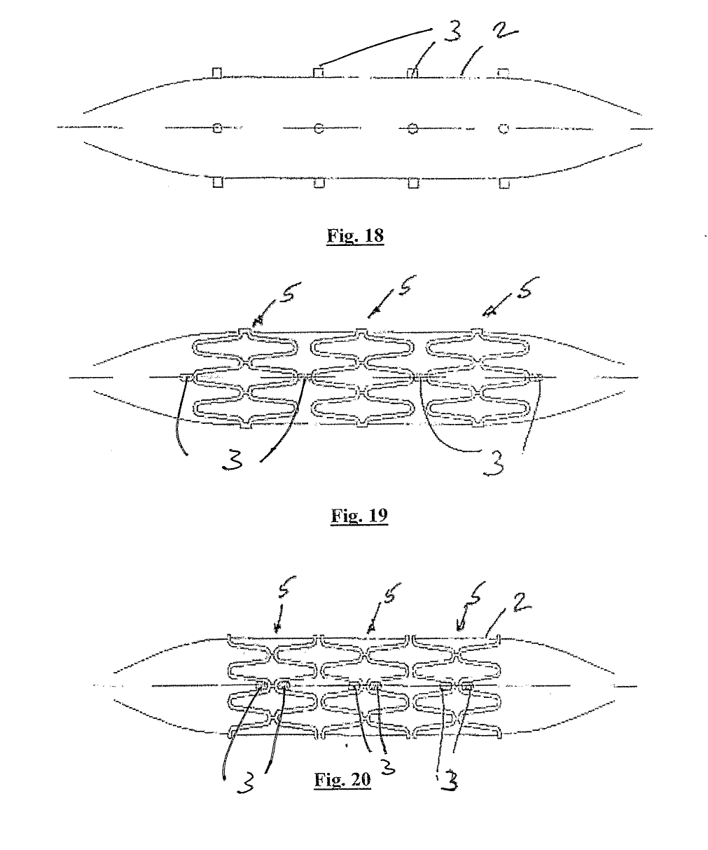

Referring to FIG. 17 another possibility of coupling a stent 5 with the balloon 1 is illustrated. In this case there is a cylindrical, or other shaped, ridge 3 on the balloon surface which is designed to be inserted into an appropriately designed hole 16 in one or more of the stent struts. The hole 16 in the stent strut would create either a tight or loose bond with the balloon ridge. A loose bond is preferable to insure the separation of the stent 5 from the balloon 1 once the stent 5 has been expanded and the balloon 1 is being retracted. FIG. 17 illustrates a single-cell stent 5 coupled onto a balloon 1 through an appropriately designed cylindrical ridge 3 on the balloon surface and a corresponding hole 16 in the connecting strut between two segments, forming a loose bond. The number coupling points around the balloon/stent circumference can be one or more, any may, most likely correspond to the number of folds in the balloon 1.

Referring to FIG. 18 there is illustrated a variation on the design in which the ridges 3 are predominantly cylindrical, or elliptical. This allows a better conformity between the stents and the ridges in the crimped configuration.

FIG. 19 illustrates an example of the coupling of mini-stents 5 to a balloon 1 and its ridges 3. In this case, three single-cell mini-stents 5 are coupled to a balloon 1 with two axial sets of four ridges 3 (hatched) at 180° circumferential spacing. The ridges 3 are positioned in a manner so that each of the mini-stents 5 is coupled to four ridges 3 at four separate locations (two of which are on the part of the balloon that is not visible). Each of the coupling locations is on the outer maximum points of the mini-stent edges. Such a configuration provides sufficient axial support and position control. The mini-stents 5 may be designed in such a way that they substantially conform to the balloon ridges 3 in the crimped configuration. In this balloon/stent configuration, the wall coverage after deployment will be low due to the large gaps between individual mini-stents 5. In other embodiments the mini-stents 5 may be designed to be positioned out-of-phase and closer together in a manner for them to interleave. The spacing between axial ridges is in this case equal.

Referring to FIG. 20 there is illustrated a balloon 1 in which the mini-stents 5 are coupled at the base of the apex. Here, the axial spacing between ridges 3 is not equal. In other embodiments, the coupling point may be located at an intermediate point with correspondingly designed stents 5 to accommodate the coupling.

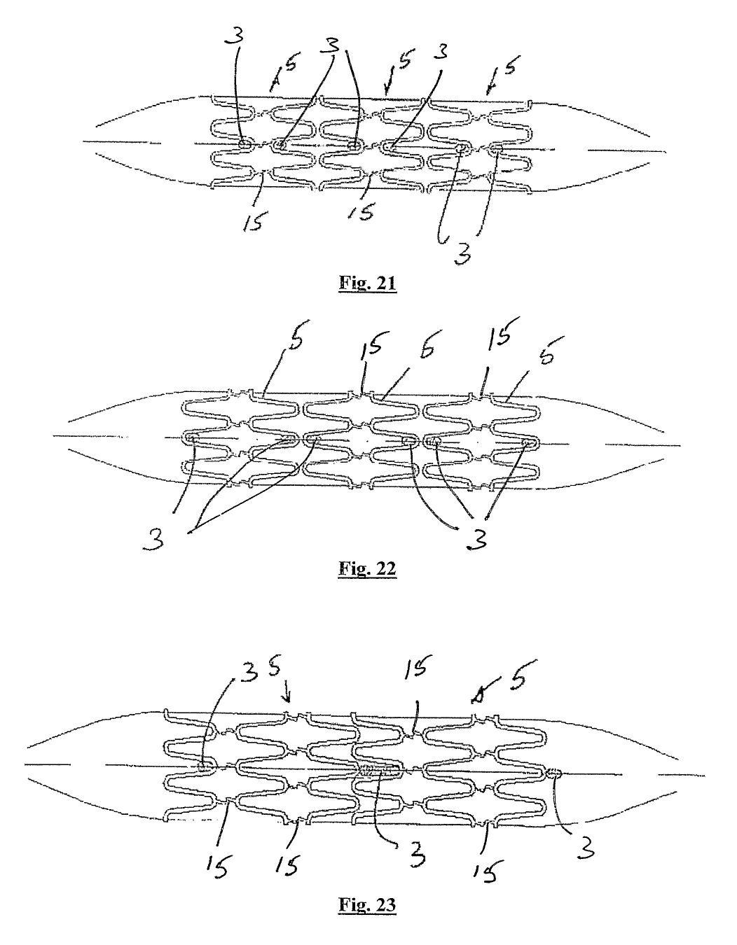

FIG. 21 illustrates another embodiment of a balloon/mini-stent coupling. In this case, the main difference lies in the design of the stent 5. A connecting strut 15 between two segments of the stent 5 is axially flexible so as to compensate for any foreshortening or elongation caused by the balloon/mini-stent coupling. Many different stent designs are possible.

FIG. 22 illustrates another embodiment of a balloon/mini-stent coupling. The mini-stent 5 is coupled at the base of the opposite apex illustrated at FIG. 20. This figure illustrates how the use of ridges can be used to ensure link elements straighten during expansion, as in the absence of constraining ridges the stents may foreshorten due to longitudinal contraction of the stents and the link elements will not elongate. This may leave large unsupported gaps between stents.

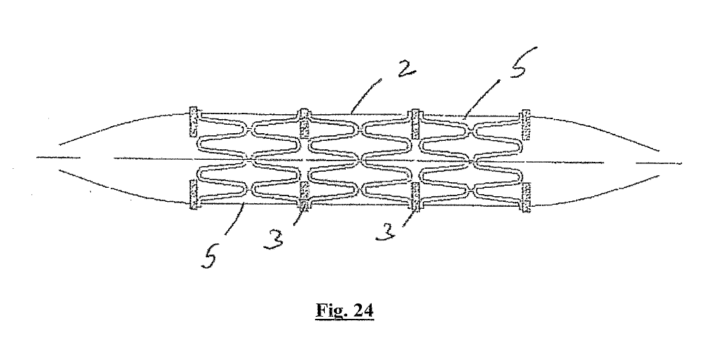

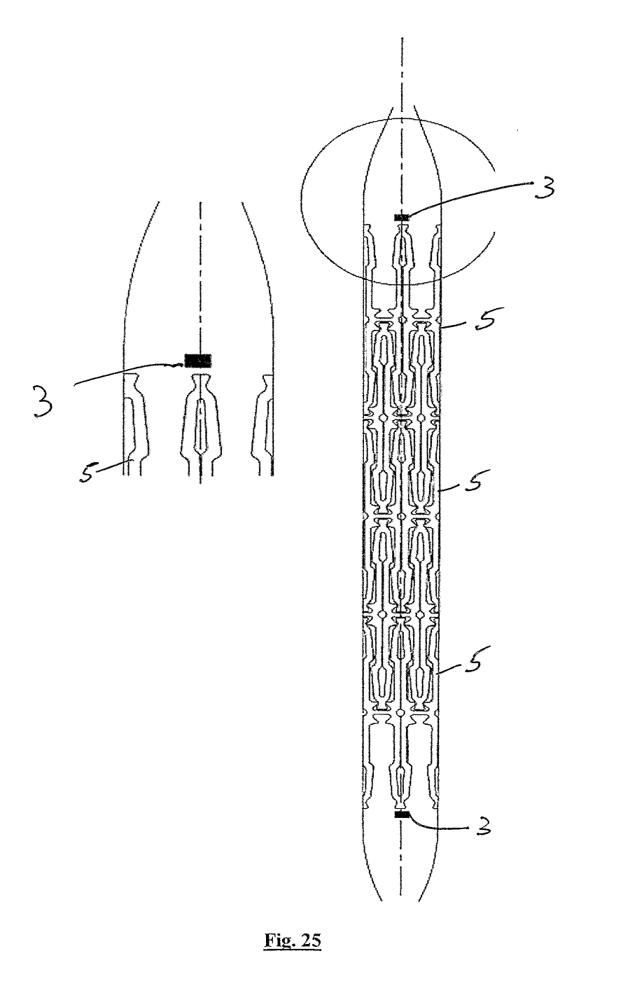

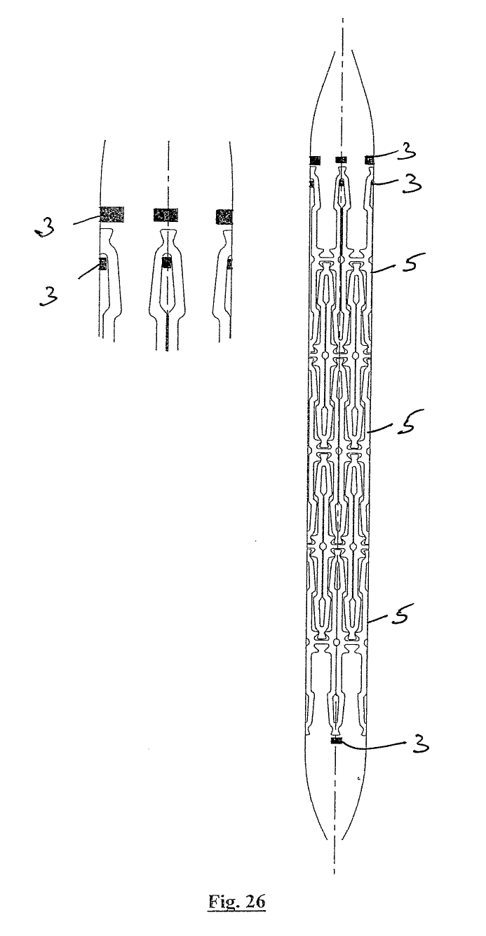

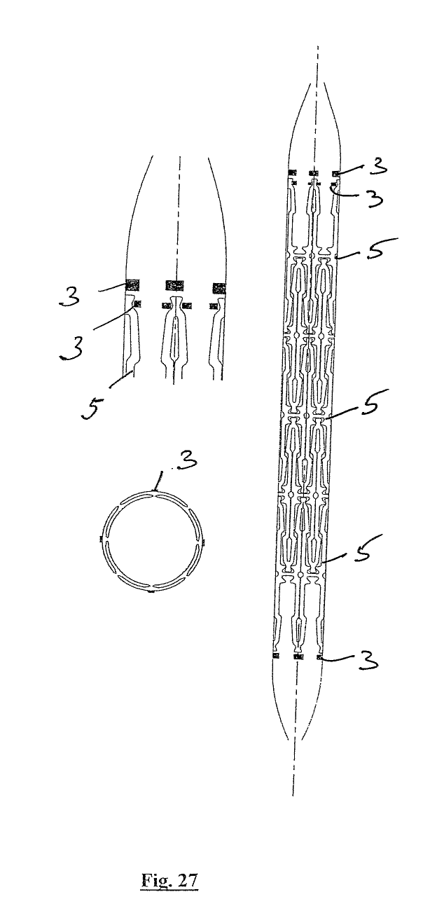

Referring to FIG. 23, in this case each stem segment, or mini-stent 5, consists of more than one cell in length. The stents 5 used with the balloon 1 may be of varying lengths, in terms of the number of segments. In the case of FIG. 23, there are two sets of three ridges 3 on the balloon surface. The outer ridges 3 are smaller than the central ones to facilitate coupling. In this case, there is also a degree of interleaving between mini-stents 5 to provide improved coverage. En reality, the degree of interleaving may be much larger than illustrated.

In the embodiment illustrated in FIG. 24 three single-cell mini-stents 5 are mounted on a balloon 1 with three sets of four circumferential ridges 3.

FIG. 25 is a side view of multiple interlocking stents crimped onto a single balloon constrained by ridges at the ends of the balloon to prevent axial movement of the stent along the balloon. Any type of balloon folding pattern can be used in this case.

FIG. 26 is a side view of the multiple interlocking stent segments with the formations controlling the movements of the terminal segments. Rotational movements of the stents are controlled by the ridges. If the ridges are placed on RE spots, the stents will not move circumferentially on the balloon during expansion. The outer formations limit axial (longitudinal) stent movement in one direction, and the inner formations limit movement in the opposite direction. The inner formations also control the rotation of the terminal segments relative to the balloon. The opposite terminal stent is controlled by only one set of ridges, but any combination of ridges may be used to achieve the desired function.

FIG. 27 is a side view of the interlocked multiple stents mounted in a SOF balloon with formations at the ends. The outer and inner formations control the movement of the terminal segments. The ridges are placed at RE spots. The inner ridges also control the relative rotation of the stents with respect to the balloon.

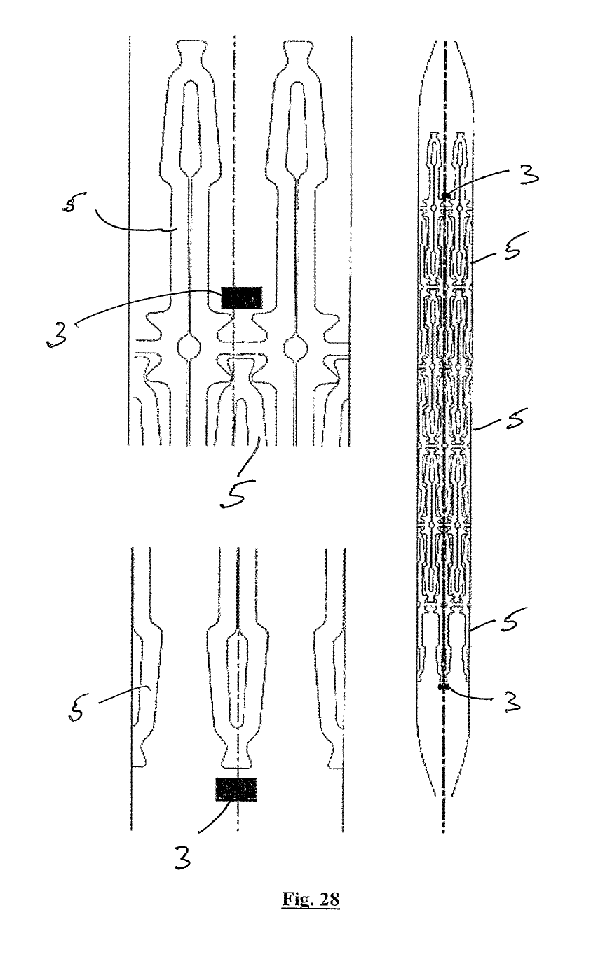

FIG. 28 is a side view of multiple interlocking stents with different locations for the balloon formations. One is located at the end of the terminal stent segment and the other one at the middle of the opposite terminal segment. The ridges may be placed on the RE spots.

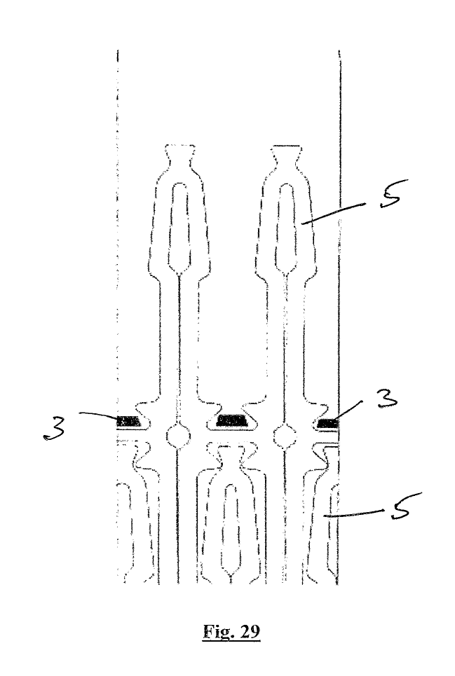

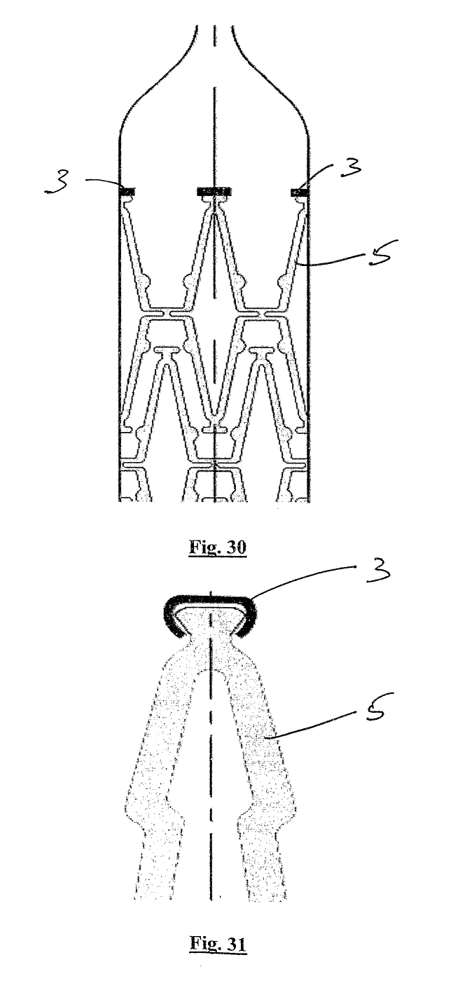

FIG. 29 is a side view of multiple interlocking stents crimped onto a balloon with ridges acting to constrain the stent segment until such a point in the expansion process where the stent opens sufficiently to move past the ridge. The position of the ridge ensures foreshortening about the centre of the stent segment.

FIG. 30 illustrates an alternative interlocking stent system where the stents will disengage earlier in the expansion process and where the ridges on the balloon control axial stent movement.

FIG. 31 is a balloon formation to limit the movement of the end of the stent segment circumferentially and longitudinally with respect to the balloon. This configuration will also limit circumferential movement of the stent if the ridges are located at RE spots.

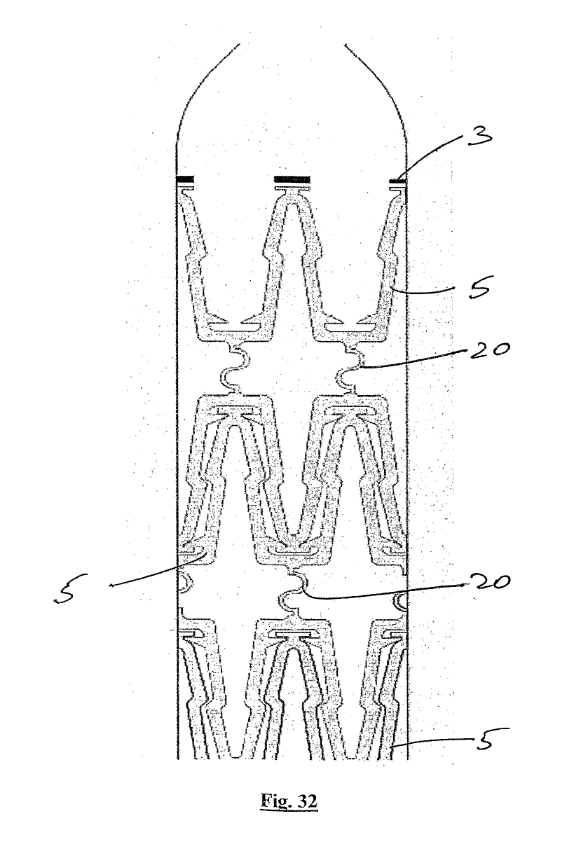

FIG. 32 is a side view of multiple interlocking stents crimped onto a single balloon constrained by ridges at the ends of the balloon to prevent axial movement of the stent along the balloon, where the stent segments have link elements 20 in their body that may straighten during expansion and hence limit foreshortening.

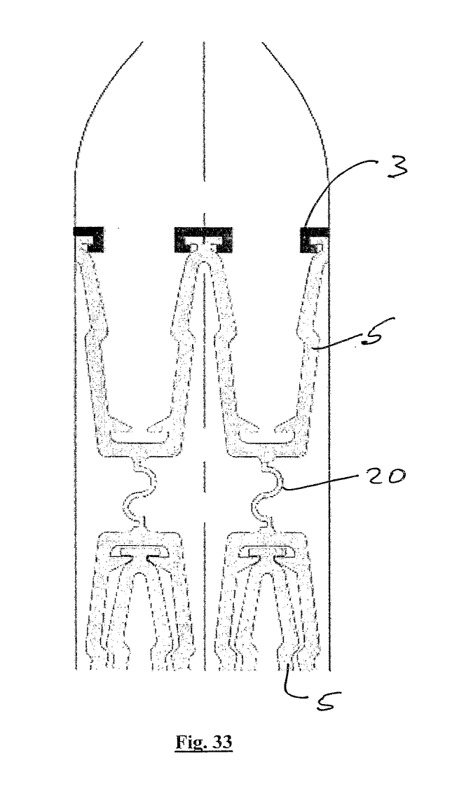

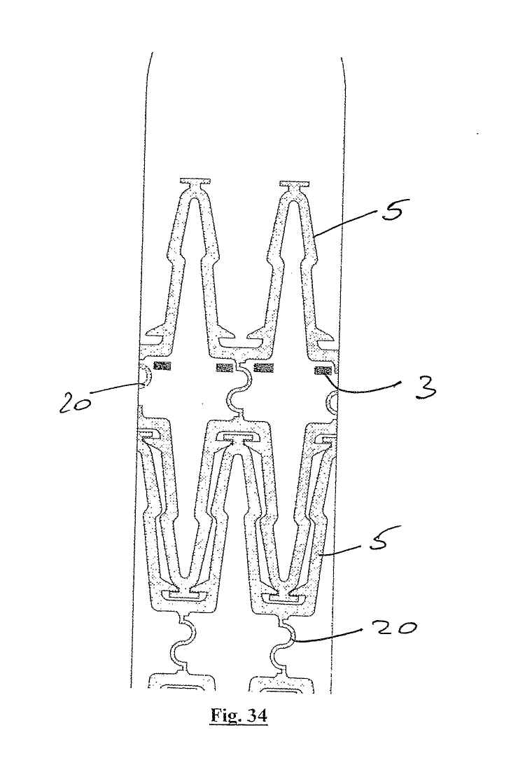

FIG. 33 is a side view of balloon having ridges at the end to prevent the movement of the terminal segments in both directions, where the stent segments have link elements 20. Another advantage of this configuration is that the expanded length of the entire stent system is identical to the length in the crimped configuration (i.e. no overall foreshortening);

FIG. 34 illustrates an alternative position for the ridges on the balloon where the stent segments have link elements 20.

The invention facilitates deployment of a plurality of physically unconnected mini-stents at a controlled spacing, which when in contact with an artery wall, may be perceived as a single, highly flexible stent. When deployed the individual mini-stent segments will not be in contact with each other, thus allowing sufficient support of the vessel wall as well as superior flexion compared to known stents.

One potential application of the invention is in the advanced form of PAD in the limbs is Critical Limb Ischemia, and affects around 10m Americans. A stenting intervention in cases of CLI is not life saving, but if left untreated amputation can result.

The invention is not limited to the embodiment hereinbefore described, with reference to the accompanying drawings, which may be varied in construction and detail.

1-39. (canceled)

40. A stent delivery system comprising:—a plurality of separate or separable balloon expandable stent segments;
an expansible balloon on which the stent segments are mounted;
the balloon having a number of formations to control movement of the stent segments during deployment.

41. The stent delivery system as claimed in claim 40 wherein the formations control the position of the stent segments on the balloon in a delivery configuration and/or in the expanded configuration.

42. The stent delivery system as claimed in claim 40 wherein the formation engage with at least some of the stent segments during deployment.

43. The stent delivery system as claimed in claim 40 wherein the formations engage with at least some of the stent segments in the delivery configuration and/or in the expanded configuration.

44. The stent delivery system as claimed in claim 40 wherein the balloon is folded in a delivery configuration and the formations are arranged such that the formations move only in a radial direction on expansion of the balloon.

45. The stent delivery system as claimed in claim 44 wherein there are a plurality of circumferentially spaced-apart formations.

46. The stent delivery system as claimed in claim 44 wherein the balloon has a longitudinal axis and there are a plurality of longitudinally spaced-apart formations.

47. The stent delivery system as claimed in claim 40 wherein at least some of the formations are integral with the balloon.

48. The stent delivery system as claimed in claim 40 wherein at least some of the formations are mounted or mountable to the balloon.

49. The stent delivery system as claimed in claim 40 wherein at least some of the formations comprise protrusions extending radially outwardly of the balloon.

50. The tent delivery system as claimed in claim 49 wherein the protrusions extend radially outwardly of the balloon for a distance which is approximately the same order of thickness of a stent element.

51. The stent delivery system as claimed in claim 40 wherein the stent segments comprise a proximal segment, a distal segment and at least one intermediate segment.

52. The stent delivery system as claimed in claim 51 wherein the segments are inter-engaged at least in the delivery configuration.

53. The stent delivery system as claimed in claim 51 wherein formations are located on the balloon to control the movement of at least the distal stent segment.

54. The stent delivery system as claimed in claim 51 wherein formations are located on the balloon to control the movement of at least the proximal stent segment.

55. The stent delivery system as claimed in claim 51 wherein there are link elements within at least some of the segments and the protrusions constrain the proximal and distal stent segments to cause at least some of the link elements to elongate during expansion.

56. The stent delivery system as claimed in claim 51 wherein formations are located on the balloon to control the movement of at least one of the intermediate stent segment(s).

57. The stent delivery system as claimed in claim 40 wherein in the delivery configuration the formations are all on the outer surface of the balloon folds or surface.

58. The stent delivery system as claimed in claim 40 wherein the height of the formations does not change significantly during expansion.

59. The stent delivery system as claimed in claim 40 wherein the multiple stent segments are separate segments which are not interlinked.

60. The stent delivery system as claimed in claim 40 wherein the stent segments have coupling parts for coupling of the segments, the segments being movable between:—
a collapsed delivery configuration in which the coupling parts of the segments are interengaged; and
a deployed configuration in which the coupling parts are disengaged.

61. The system as claimed in claim 40 wherein the stent segment comprises a first set of strut elements and a second set of strut elements.

62. The system as claimed in claim 61 wherein the stent segment comprises a first set of one or more link elements to link at least some of the first set of strut elements to at least some of the second set of strut elements.

63. The system as claimed in claim 60 comprising a plurality of axially arranged radially expandable stent segments, the segments having coupling parts for coupling of the segments, the segments being movable between:—
a collapsed delivery configuration in which the coupling parts of the segments are interengaged; and
a deployed configuration in which the coupling parts are disengaged,
wherein the segment comprises a first set of strut elements, a second set of strut elements, and a first set of one or more link elements to link at least some of the first set of strut elements to at least some of the second set of strut elements and wherein a link element is more flexible than a strut element.

64. The system as claimed in claim 40 wherein at least part of the segment comprises a biodegradable material or a radiopaque material, there may be a coating around at least part of the segment.

65. The method for delivering a luminal prosthesis to a treatment site comprising:—
providing a delivery system as claimed in claim 40, the stent segments having coupling parts which are interengaged;
delivering the catheter to a treatment site;
radially expanding all of the stent segments at the treatment site to a partially expanded configuration in which the coupling parts of the segments remain interengaged; and
further radially expanding all of the stent segments to a deployed configuration in which all of the coupling parts of the stent segments are disengaged.
